(19) United States
(12) Patent Application Publication
(10) Pub. No.: US 2026/0256587 A1
(43) Pub. Date: Sep. 3, 2026

(54) TRANSCATHETER MITRAL ANNULOPLASTY SYSTEMS, DEVICES AND METHODS

(71) Applicant: Medtronic, Inc., Minneapolis, MN (US)

(72) Inventors: Emily A. Grimm, Petaluma, CA (US); Maria E. Valdovinos, Santa Rosa, CA (US); William A. Berthiaume, Santa Rosa, CA (US); Fatemeh Fatemi Far, Richmond, CA (US); Karan P. Punga, San Rafael, CA (US); Matthew E. Genovese, Windsor, CA (US)

(73) Assignee: Medtronic, Inc., Minneapolis, MN (US)

(21) Appl. No.: 19/656,404

(22) Filed: Apr. 23, 2026

Related U.S. Application Data
(63) Continuation of application No. 17/708,388, filed on Mar. 30, 2022.
(60) Provisional application No. 63/184,470, filed on May 5, 2021.

(51) Int. Cl. A61F 2/24

(52) U.S. Cl. CPC A61F 2/2466; A61F 2/2445; A61F 2220/0008; A61F 2220/0075

(57) ABSTRACT

Aspects of the disclosure include systems and devices for delivering an annuloplasty implant and anchor delivery apparatus to a valve annulus. The systems and devices of the disclosure are configured to dispense and secure or anchor the implant to the valve annulus. Various implants of the disclosure include one or more cinching members that can be tensioned to reshape the valve annulus. Methods of deploying the implant, securing the implant to the valve annulus and reshaping the valve annulus are also disclosed.

### CROSS-REFERENCE TO RELATED APPLICATION

This Non-Provisional Patent Application is a continuation of U.S. Non-Provisional patent application Ser. No. 17/708,388, filed Mar. 30, 2022, which claims the benefit of the filing date of U.S. Provisional Patent Application Ser. No. 63/184,470, filed May 5, 2021, the entire teachings of both of which are incorporated herein by reference.

### FIELD

The present technology is generally related to delivery systems, annuloplasty devices and methods of reducing a valve annulus for treatment of mitral regurgitation, for example.

### BACKGROUND

Generally, the anatomy and physiology of the human heart is well known. Of the four one-way valves in the heart, the two inlet valves are the mitral valve of the left side of the heart, and the tricuspid valve on the right side of the heart. The tricuspid valve is located between the right atrium and the right ventricle. The three leaflets of the tricuspid valve laterally terminate at the tricuspid annulus. Blood flows from the superior and inferior vena cava into the right atrium, then through the tricuspid valve during diastole to fill the right ventricle. During ventricular systole, the tricuspid valve is closed and blood is ejected through the pulmonary valve into the pulmonary artery and hence through the lungs. At the end of ventricular systole the pulmonary valve closes. Leaving the lungs, the now oxygenated blood flows into the left atrium and hence through the mitral valve into the left ventricle during ventricular diastole. Finally, at ventricular systole the mitral valve closes and blood is ejected through the aortic valve into the aorta. However, should the mitral valve become regurgitant due to disease then some percentage of the left ventricular stroke volume will flow backwards through the mitral valve into the left atrium. This regurgitation causes the left atrial pressure to rise, in turn causing pulmonary artery pressure to rise, which is reflected back to the right ventricular pressure.

Typically, to treat a patient with functional mitral regurgitation, a physician places an annuloplasty ring on the mitral annulus to reduce the circumference and septal-lateral diameter of the annulus. In degenerative mitral regurgitation patients, annuloplasty rings are utilize to stabilize the mitral annulus, not reduce the annular circumference.

The present disclosure addresses problems and limitations associated with the related art.

### SUMMARY

The techniques of this disclosure generally relate to systems and methods of reducing a valve annulus for treatment of mitral regurgitation, for example. Aspects of the disclosure provide delivery systems that can cinch an implant (e.g., annuloplasty ring) from two ends to accomplish a substantially symmetrical or symmetrical cinching or reshaping of the valve annulus.

In one aspect, the present disclosure provides a system comprising a delivery device including a guide catheter defining a lumen. The guide catheter housing an implant catheter and an anchor catheter at least partially within the lumen, and an anchor delivery apparatus slidably positioned within the anchor catheter. The system further includes an implant positioned within the implant catheter and engaged with a distal end of the anchor catheter.

In another aspect, the disclosure provides a system comprising a guide catheter defining a first lumen and a second lumen and an implant extending at least partially within the first lumen. The system further includes an anchor delivery apparatus positioned within the second lumen.

In another aspect, the disclosure provides a system includes a guide catheter defining a guide catheter lumen. Additionally, the system includes an anchor catheter having a body defining an anchor catheter lumen terminating at a distal opening at a distal end of the anchor catheter. The system also includes an anchor delivery apparatus at least partially positioned within the anchor catheter and maintaining a plurality of anchors. A fabric implant extends along the body of the anchor catheter. The implant has a distal end that is transversely oriented with respect to the distal opening of the anchor catheter lumen. One of the plurality of anchors is engaged with the implant.

In an additional aspect, the disclosure provides a system including a catheter having a first lumen and a second lumen. The system has an implant positioned within the first lumen and an anchor delivery apparatus provided in the second lumen. The anchor delivery apparatus is configured to deliver a plurality of anchors into the implant as the implant exits the first lumen.

In another aspect, the disclosure provides a method of repairing a valve annulus including providing a system including a guide catheter. An implant is positioned at least partially within the guide catheter and an anchor delivery apparatus is positioned within the guide catheter. The method includes directing a distal end of the guide catheter to a valve annulus, advancing the implant at least partially out of the guide catheter and securing the implant to a valve annulus with the anchor delivery apparatus. The method further includes advancing the implant out of the guide catheter and further securing the implant to the valve annulus with the delivery apparatus.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a partial, schematic, perspective view of one delivery system of the disclosure.

FIG. 1B is an end view of the delivery system of FIG. 1A further including an optional outer catheter.

FIG. 2 is a perspective view of the delivery system of FIGS. 1A-1B dispensing and anchoring an implant to a valve annulus.

FIGS. 3A-3F are additional views of the delivery system of FIGS. 1A-2 dispensing and anchoring the implant to the valve annulus.

FIG. 4 is a top view of the implant fully secured to the valve annulus.

FIGS. 5A-5M illustrate various cinching methods of implants of the disclosure to resize the valve annulus.

FIG. 6A is a top view of a cinching device.

FIG. 6B is a perspective view of the cinching device of FIG. 6A.

FIG. 7 is a top view of the cinching device of FIGS. 6A-6B having a cinching member positioned within the cinching device.

FIG. 8A is a cross-sectional view of the cinching device of FIGS. 6A-7 operatively connected to an end of the implant for proximally pulling (i.e. tensioning or cinching) the cinching member(s).

FIGS. 8B-8F schematically illustrate catheter features utilized to adjust a nut of the cinching device of FIG. 8A.

FIG. 9 schematically illustrates the catheter of FIGS. 8A-8F adjusting the implant with the cinching device of FIGS. 6A-8F.

FIG. 10A is a top view of an alternate cinching device.

FIG. 10B is a perspective view of the cinching device of FIG. 10B.

FIG. 11 is a perspective view of an alternate cinching device configured to engage a nut connected to the cinching member.

FIG. 12 is a schematic illustration of the cinching device of FIG. 11 at the implant and valve annulus for proximally pulling the cinching member(s).

FIG. 13 is a perspective view of the implant including the cinching member (not visible) having a first threaded end.

FIG. 14 is a perspective view of the implant and cinching member of FIG. 12 having a first nut engaged with the first threaded end.

FIG. 15 is a perspective view of the implant and cinching member of FIG. 12 having a second nut engaged with the second threaded end.

FIG. 16 is a perspective view of the first threaded end after being drawn through the first nut to tension the cinching member(s).

FIG. 17 is a schematic view of the implant secured to the annulus after cinching.

FIG. 18 is a schematic illustration of one end of the implant having a support, wherein the second end can be identically configured.

FIG. 19 is a perspective view of the support of FIG. 18.

FIG. 20 is a schematic illustration of the support of FIGS. 18-19 stitched within a lumen of the implant defined by a stitch extending through the body, and along a length of the implant.

FIG. 21A is a partial, side view of an alternate delivery device of the disclosure.

FIG. 21B is a partial, side view of the delivery device of FIG. 21A in a partially-deployed arrangement.

FIGS. 21C-21E are a partial, side view of the delivery device of FIG. 21B illustrating a collar.

FIG. 21F is a partial, side view of the delivery device of FIGS. 21A-21B in a deployed arrangement.

FIG. 22A is a partial, side view of an alternate delivery device having a body defining a main lumen and a first lumen to house an implant.

FIG. 22B is a partial, side view of the delivery device of FIG. 22A having an anchor catheter extending from the main lumen.

FIG. 22C is a partial, perspective view of the delivery device of FIGS. 22A-22B omitting the optional anchor catheter.

FIG. 23 is a partial, perspective view of an alternate delivery device.

FIG. 24A is a partial, perspective view of an alternate delivery device having a body.

FIG. 24B is a partial, top view of the delivery device of FIG. 24A having the body shown as partially cut away to show the path of cinching and elongated members.

FIG. 25 is a front view of a modified outer catheter having a cutout.

FIG. 26 is a front view of a modified outer catheter, similar to that of FIG. 25 but having an alternate cutout.

FIG. 27 is a partial, perspective view of a catheter having a guide configured to receive the implant.

FIGS. 28-30 are perspective views of collars of the disclosure.

FIG. 31 is a cross-sectional view of a catheter configuration having a lumen configured to house an imaging apparatus.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

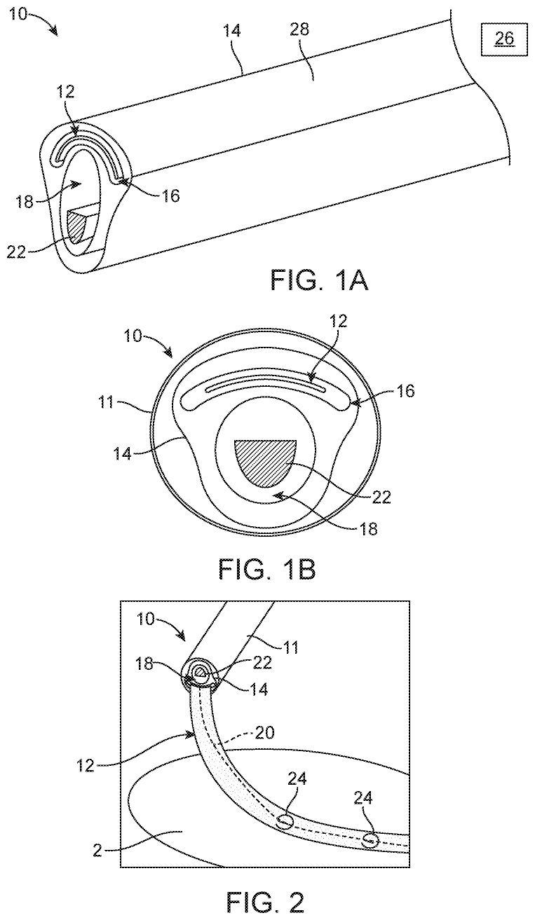

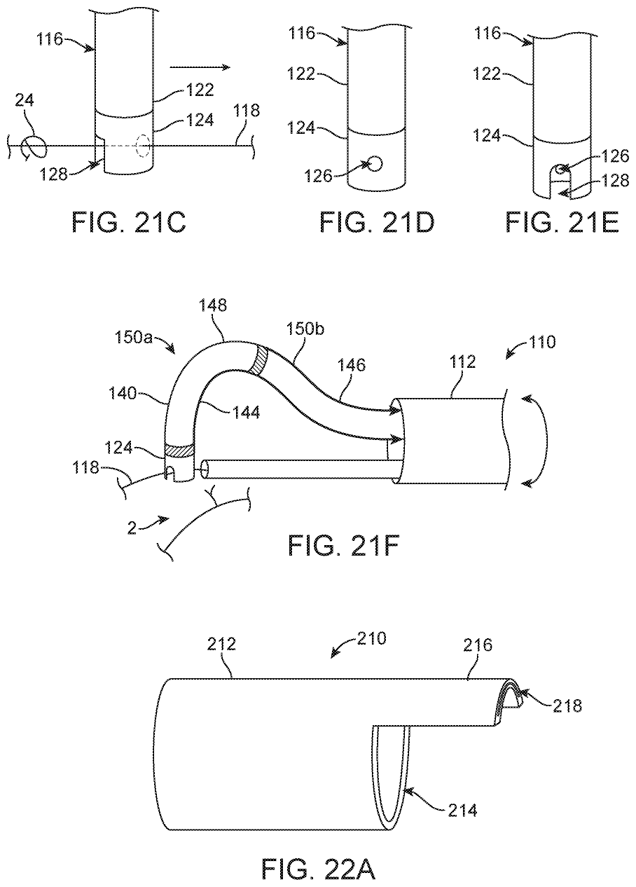

Select components of an illustrative example of a delivery system 10 are collectively illustrated in FIGS. 1A-4. This delivery system 10 is particularly suitable for cloth implants 12 or the like. The delivery system 10 includes a guide catheter 14 having first and second lumens 16, 18. Optionally, the guide catheter 14 is housed in an outer catheter 11 (only shown in FIG. 1B), similar to catheter 112 of FIG. 21A. The outer catheter 11 provides a cylindrical outer surface to mitigate whipping when the outer catheter 11 (housing guide catheter 14) is positioned within the septal fossa (which requires a ~90 degree turn) and when the outer catheter 11 steers down to, and round, the annulus 2 (requiring another ~90 degree turn, plus additional curving). In other embodiments, the guide catheter 14 can be configured to define a cylindrical outer surface. Such an outer catheter 11 can be configured to be torqueable. In one illustrative example, the guide catheter 14 is at least partially made of a braided material. In one example, the first lumen 16 has an extruded crescent shape and houses the implant 12. The extruded crescent shape allows for the implant 12 to be loaded into and move within the first lumen 16 in the orientation that it will be applied to a heart valve annulus 2.

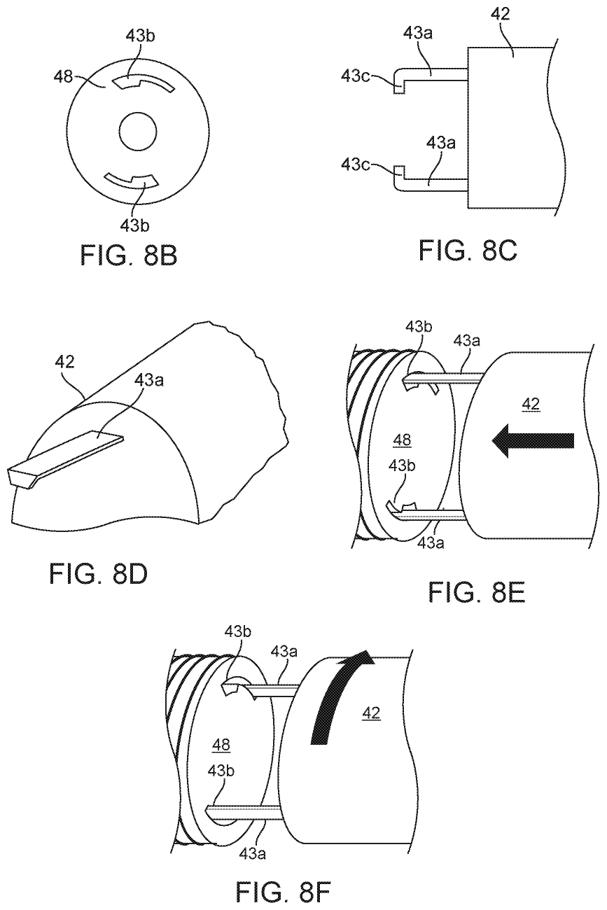

For example, in some embodiments, the implant 12 is an unfolded, rectangular cloth implant that is positioned within the first lumen 16 in an unfolded arrangement. In another example, the implant 12 may include a rectangular cloth material having a single fold, optionally defining a lumen or passageway 15 (FIG. 8A) for one or more elongated cinching members 20 or other structural members, such as wires, cords, sutures, filaments or the like. The implant 12 can include additional lumens formed by stitched seams, or otherwise.

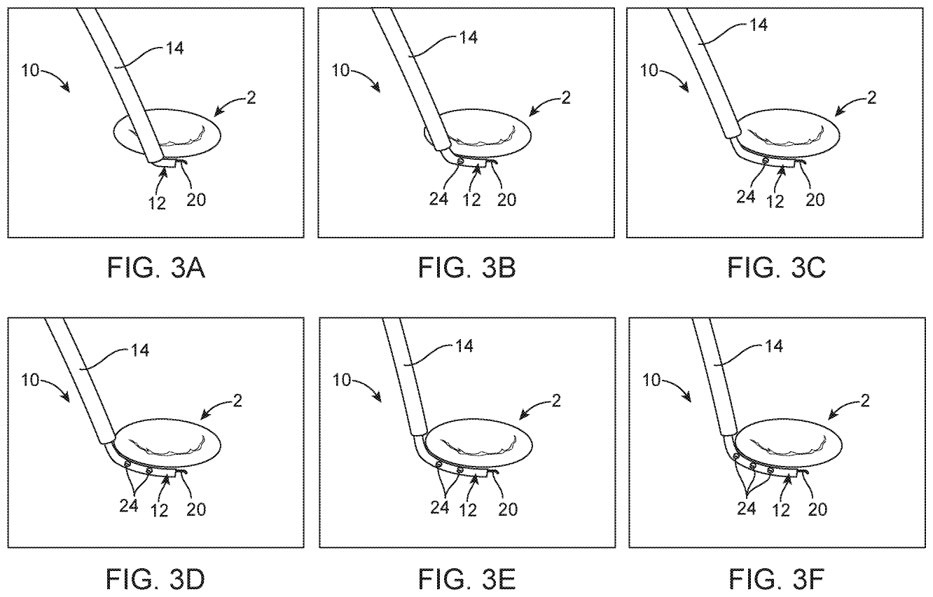

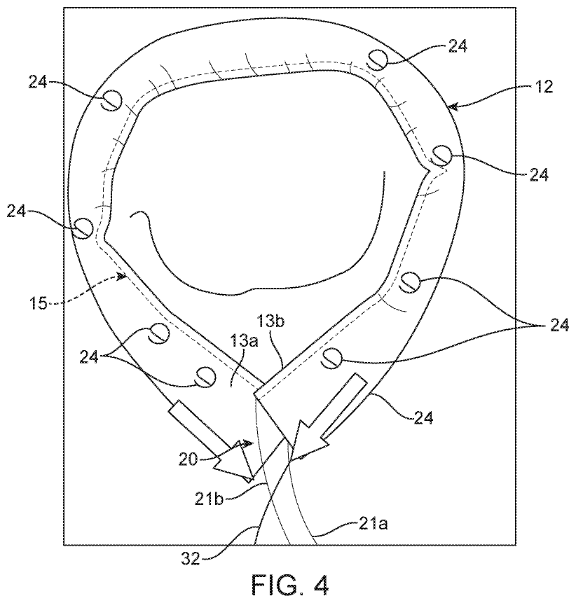

The second lumen 18 can be cylindrically shaped and houses an anchor delivery apparatus 22. The anchor delivery apparatus 22 can be configured to deliver of one or more anchors 24 to secure the implant to the annulus 2. In one example, each anchor 24 is applied between two lumens formed by implant 12. The anchor delivery apparatus 22 can be of any known device for implanting annuloplasty anchors. Non-limiting examples include applier model numbers SG-64, HG-16-62-28, SA-85, HG-19-90-22, HG-18-90-32, HG-18-90-42 or HA-18-114 available from Medtronic, Inc. of Minneapolis, Minnesota, for example. Such an anchor delivery apparatus 22 is configured to delivery one or more anchors 24 (e.g., 6-14 anchors) through the implant 12 and into the annulus 2, sequentially delivering anchors 24 around the annulus 2 until the implant 12 is secured at a plurality of locations along a length of the implant 12 as is generally shown in FIGS. 3A-4. Anchors 24 can be any type of known surgical anchor suitable for securing an implant to a heart valve annulus. Some non-limiting examples are helical, double helical, tapered helical, “push” style anchors, or the like. In one non-limiting example, the anchors are delivered starting at a lateral trigone, travelling along a posterior annulus, and ending at medial trigone (or vice versa), for example. As desired, the most recently deployed anchor can be dislodged from the annulus and repositioned. The present inventors have found that, generally, once the subsequent anchor has been implanted into the annulus, the previous anchor cannot practically be repositioned without removing the anchors that are between the delivery catheter and the anchor to be removed.

Because the implant 12 is maintained within the first lumen 16 of the guide catheter 14 in the arrangement in which the implant 12 will be applied to the annulus 2, the delivery system 10 is configured to deliver anchors 24 at a midpoint of the width of the implant 12 when the implant 12 is released from the first lumen 16 (see FIGS. 3F-4). In this embodiment, the guide catheter 14 can either be torqueable via a handle assembly 26 (schematically depicted in FIG. 1A) and/or can be configured to include steering pull wires secured to the guide catheter 14 to allow for two planes of steering with or without torqueing abilities in a manner that that is discussed in greater detail below with respect to FIG. 21F. Additional steering capabilities of the system 10 can be utilized. In various examples, the outer catheter 11 and the guide catheter 14 can be configured to have one plane of steering of the type utilized in the art. The configuration of the embodiment of FIGS. 1A-2 is particularly advantageous as the fixed positioning between the first and second lumens 16, 18 aids in maintaining the implant 12 with respect to the anchor delivery apparatus 22 as the guide catheter 14 clocks or rotates, around the annulus 2.

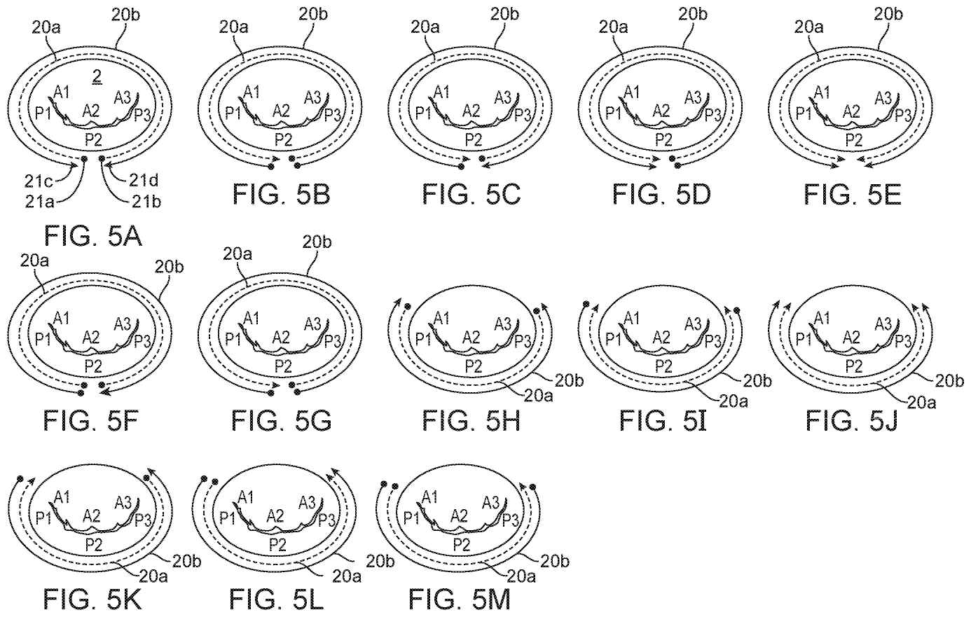

In various examples, the implant 12 includes at least one cinching member 20a extending within the fold of the implant 12. The cinching member 20a can be made of aramid fiber yarn, ultra-high molecular weight polyethylene sutures, braided flat stainless steel wire, stainless steel wire or NiTi wire. Each cinching member 20a has a first end 21a and a second end 21b. After the implant 12 is positioned around and anchored around the annulus 2 as depicted in FIGS. 3A-4, the first and/or second ends 21, 21b can be tensioned proximally (FIG. 4) to cinch and reshape the implant 12 and annulus 2 secured thereto. The anchored implant 12, and thus the annulus 2 secured thereto, can be cinched in many ways to result in the desired annulus shape. Various cinching options are schematically illustrated in FIG. 5A-5M. In these figures, only the cinching members 20a, 20b are illustrated and other aspects of the implant and system are omitted for clarity. Arrows are used on various ends 21a, 21b, 21c, 21d of the cinching members 20a, 20b to illustrate a direction of cinching to tensioning whereas ends 21a, 21b, 21c, 21d including dots indicate a fixation point of the respective cinching member to the annulus. In various examples, the implant may include two cinching members 20a, 20b including an inner cinching member and an outer cinching member. The disclosure is not limited to any particular method or number of cinching members.

In the example of FIG. 5A, the inner cinching member 20a has one end 21a fixed at P2 and the inner cinching member 20a extends around the annulus 2 from P2 to P1 (i.e. anterior or medial scallop) to P3 (i.e. posterior scallop or lateral scallop) and the second end 21b is fixed at P2 (i.e. middle scallop), proximate the first end 21a. The outer cinching member 20b extends around the annulus 2 and includes first and second ends 21c, 21d extending out of the implant (not shown) at P2, which can both be tensioned or pulled proximally at P2. Only FIG. 5A is fully referenced for ease of illustration. It is to be understood that identically named elements of FIGS. 5B-5M include identical cinching members with the exception of placement around the annulus 2 and in that the respective ends 21a, 21b, 21c, 21d vary in attachment to the annulus.

In the example of FIG. 5B, the inner cinching member has the second end fixed at P2 and extends around P3 to P1 and to P2 where the first end exits the implant and can be tensioned proximally. The outer cinching member extends around the annulus and includes first and second ends secured to the annulus at P2.

In the example of FIG. 5C, the inner cinching member has the second end fixed at P2 extends from P2 to P3 to P1 and to P2 where the first end exits the implant and can be tensioned proximally. The outer cinching member has the first end secured to the annulus at P2 and extends from P2 to P1 to P3 to P2, where the second end exits the implant and can be tensioned.

In the example of FIG. 5D, the inner cinching member has the second end fixed at P2 and extends from P2 to P3 to P1 and to P2 where the second end exits the implant and can be tensioned proximally. The outer cinching member has the second end secured at P2 and extends from P2 to P3 to P1 to P2, where the first end exits the implant and can be tensioned proximally.

In the example of 5E, the inner cinching member includes the first end secured to the implant at P2 and then extends from P2 to P1 to P3 and then to P2, where the second end can exit the implant and be tensioned proximally. The outer cinching member extends from P2 to P1 and P3 and then to P2, such that the first and second ends of the outer cinching member are not fixed to the annulus and can be proximally tensioned at P2.

In the example of FIG. 5F, the inner cinching member includes first and second ends secured at P2, the inner cinching member extending from P2 to P1 to P3 and to P2. The first end of the outer cinching member is attached at P2 and extends from P2 to P1 to P3 to P2 wherein the second end exits the implant and can be tensioned proximally.

In the example of FIG. 5G, the second end of the inner cinching member is secured at P2. The inner cinching member extends from P2 to P3 to P1 to P2 where the first end of the inner cinching member exits the implant and can be proximally tensioned. The first end of the outer cinching member is secured at P2. The outer cinching member extends from P2 to P1 to P3 and then to P2 where the second end exits is fixed to the annulus.

In the example of FIG. 5H, the first end of the inner cinching member is secured at P1. The inner cinching member extends from P1 to P2 to P3 where the second end is secured to the annulus. The outer cinching member has a first end extending from the implant at P1. The outer cinching member extends from P1 to P2 to P3 where the second end exits the implant for tensioning along with the first end.

In the example of FIG. 5I, the inner cinching member has a first end extending from the implant at P1. The inner cinching member extends from P1 to P2 to P3 where the second end exits the implant for tensioning along with the first end. The outer cinching member is fixed at P1 and extends from P1 to P2 to P3 where the second end is secured to the annulus.

In the example of FIG. 5J, both of the inner and outer cinching members have free first and second ends. The first ends exit the implant and are positioned at P1 and the second ends exit the implant and are positioned at P3. The first and second ends can be tensioned to reshape the implant.

In the example of FIG. 5K, the second end of the inner cinching member is fixed at P3 and extends from P3 to P2 to P1 where the first end exits the implant for tensioning. The first end of the outer cinching member is fixed at P1 and extends from P1 to P2 to P3 where the second end exits the implant for tensioning.

In the example of FIG. 5L, the first end of the inner cinching member is fixed at P1 and extends from P1 to P2 to P3 where the second end is fixed to the annulus. The first end of the outer cinching member is fixed at P1 and extends from P1 to P2 to P3 where the second end exits the implant for tensioning.

In the example of FIG. 5M, the first ends of the inner cinching member is fixed at P1 and extends from P2 to P2 to P3 where the second end exits the implant for tensioning. The outer cinching member is fixed at P1 and extends from P1 to P2 to P3 where the second end is fixed to the annulus.

Embodiments of the disclosure are particularly beneficial in that they enable symmetrical cinching of the implant 12 and annulus 2 anchored thereto. Systems and methods that cinch from one side of the implant overload forces in the anchors closest to the cinching device and can cause uneven cinching and one or more anchors to dislodge from the tissue. With asymmetric cinching techniques, more cinching often occurs further from the cinching device (i.e. where the cinching or tensioning force is applied) and less cinching is accomplished further from the cinching device. To mitigate these undesired effects, additional anchors may be placed close to the cinching device, which can result in additional undesired effects as cinching often occurs at a trigone where mitral annular dilation often occurs in the P2 region of the annulus. If a physician cinches the annulus from one side of your implant at P1 or P3 (near the valve commissures), the annular dilation might be lessened near the side of the cinching but not necessarily the other side, resulting in an asymmetrical cinch. Embodiments of the disclosure allow for symmetrical cinching of both P1 and P3 portions, toward the aorto-mitral curtain along the Y-axis, applied at P2. When the cinching force is applied at P2, along the Y-axis, the force vector includes an X component, drawing the P3 and P1 annular sections closer to one another, reducing a maximum distance between P3 and P1.

Embodiments of the disclosure are also beneficial in that the implant 12 can be oriented to resemble a full (360 degree) annuloplasty ring either pre or post cinching via tensioning of one or more cinching members 20a, 20b. For example, ends 13a, 13b of the implant 12 can be positioned to overlap or may be abutting or otherwise touching. In one example, a physician can begin anchoring the implant 12 at a trigone or at the P2 portion of the annulus 2. With a guidewire 32 (FIG. 4) extending through the lumen of the implant 12 a physician can find the ends 13a, 13b of the implant 12 after all of the anchors 24 have been deployed into tissue, regardless of where the ends 13a, 13b of the implant 12 are positioned. In this example, the implant 12 may not be connected at P2 but the anchors 24 can be placed close to each other to mitigate any further dilation of the P2 annular section. In this example, physicians can achieve either a symmetric cinch or asymmetrical cinch, as desired to treat a particular heart valve.

Once the implant 12 is resized, as desired, the position of the first and second ends 21a, 21b, 21c, 21d of the cinching member(s) can be maintained in position to effectively lock the implant in positon. In various embodiments, the cinching members (e.g., 20a, 20b) extend through a full length of the implant 12 and through the full length of the first lumen 16, such that the first and second ends (e.g., 21a, 21b, 21c, 21d) can be manipulated by the handle assembly 26 to adjust the tension and cinching. Cinching can alternatively be accomplished with a cinching device.

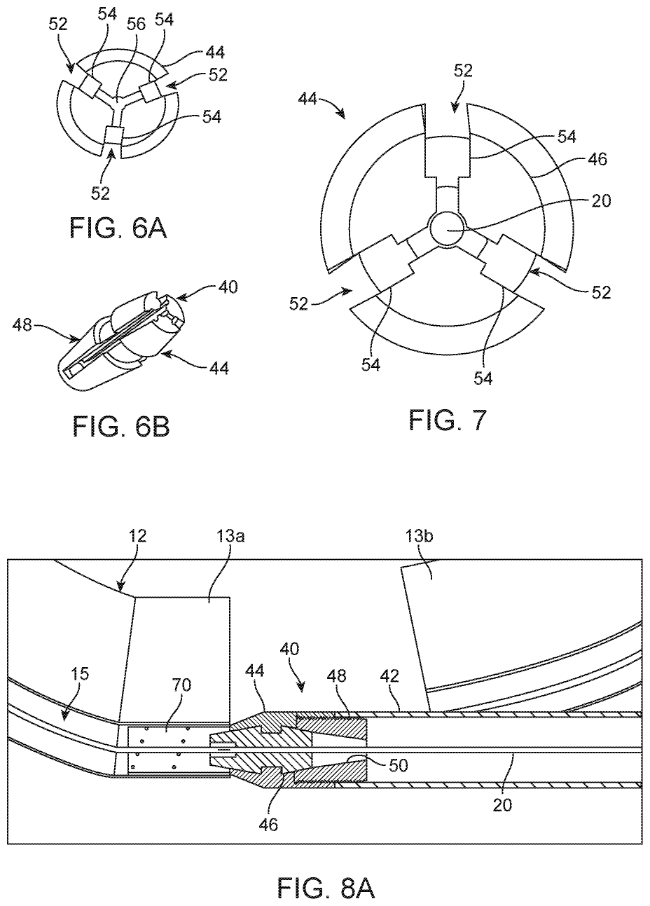

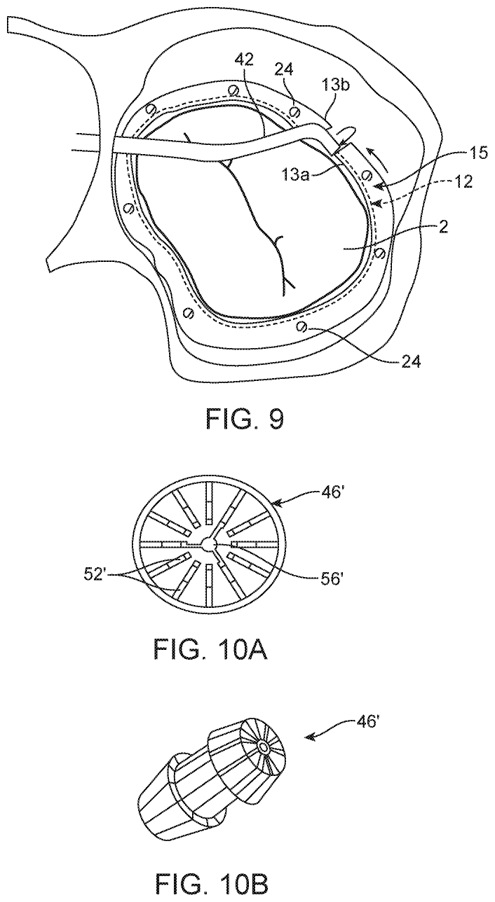

An example of one suitable cinching device 40 is collectively illustrated in FIGS. 6A-9. In this example, the cinching device 40 includes a catheter 42 (shown as transparent) and a collect holder 44 that supports a collet 46. The collet holder 44 is threadably engaged with a nut 48 having a tapered inner surface 50. The collet 46 includes a plurality of radially extending reliefs 52 adjacent a plurality of arms 54 and a central channel 56 that receives one or more cinching members 20a. The reliefs 52 allow the arms 54 to be compressed inwardly by external forces (e.g., collet holder 44), to cause an inner diameter of the central channel 56 to be reduced to compress one or more cinching members 20a threaded therethrough, thereby maintaining or locking a position of the cinching member(s) 20a. Positioning of the cinching device of FIGS. 6A-9 for cinching is generally shown in FIG. 9. In one example, catheter 42 is tracked up over the cinching member 20a (the distal end of which is external to the patient) to mate with nut 48 after implant 12 has been delivered and anchored. Although not shown in FIG. 8A, the other end of 20 would be coming out of the other end of implant 12 (the right side of the picture). In one example, two catheters 42 could be tracked up to cinch the implant simultaneously, or one 42 could be tracked up one end of the cinching member 20 (20a) and tighten, then tracked up the other end of 20 (20b) and tighten. Also to clarify, cinching member 20 is cinched from a location external to the patient; as catheter 42 abuts nut 48, tension is applied to cinching member 20, shortening the length of the cinching member 20 that resides within implant 12, thus reducing the diameter of the mitral annulus or other bodily lumen. Once the cinching member 20 has been cinched the desired amount, the catheter 42 rotates to tighten the nut 48 down onto the cinching member 20.

In some examples, it is desired to cut/snip the remaining length of the cinching member 20 once the implant 12 has been cinched sufficiently. In such examples, a “snipping” catheter could track over the cinching member 20 after the cinching member 20 has been locked in place. This snipping catheter would have a smaller diameter than catheter 42 and it would abut against the nut 48. Once in contact with the nut 48, there is a tool provided on the snipping catheter to clip the excess length of cinching member 20 so the excess length can be removed from the patient.

In one example, actuation of the nut 48 can be accomplished as follows. Catheter 42 can be configured to include features 43a that interact or interlock with nut 48. In one example, corresponding slots 43b can be provided on the proximal face of the nut 48 that are configured to receive actuator arms 43a projecting from the catheter 42. The actuator arms 43a have a tab 43c on the end that fits into slots 43b on nut 48. In the illustrated example, each slot 43b can define an opening sized to receive the tab 43b at one end but sized too small to receive the tab 43c at the second end. When the catheter 42 is rotated about its central axis, the actuator arms 43a travel through the first end of the slot 43b to the second end of the slot. When further rotation of the catheter 42 is prohibited by the slot 43b, the actuator arms 43a rotate the nut 48 to tighten the collet 46 over the cinching member 20a. The smaller end of the interlocking slot 43b is configured to ensure the actuator arm 43a cannot pull out/disengage from the nut 48 while the tightening is occurring. To release the catheter 42 from the nut 48, the catheter is rotated the opposite direction (in FIGS. 8B-8F; counterclockwise). In one example, the catheter 42 can be comprised of tightly wound coil to provide the desired torque.

The number of reliefs provided in the collet and the specific configuration of the collet can vary. For example, see FIGS. 10A-10B showing an alternate collet 46′ having 12 reliefs 52′ (generally referenced) radially positioned around a central channel 56′, which receives one or more cinching members for cinching. The collet 46′ of FIGS. 9A-9B is otherwise identical in use and operation as compared to the collet 46 of FIGS. 6A-7.

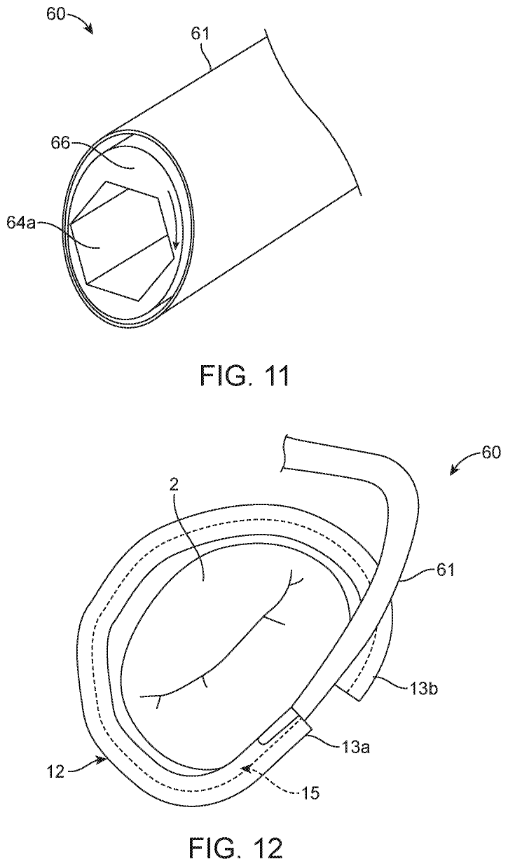

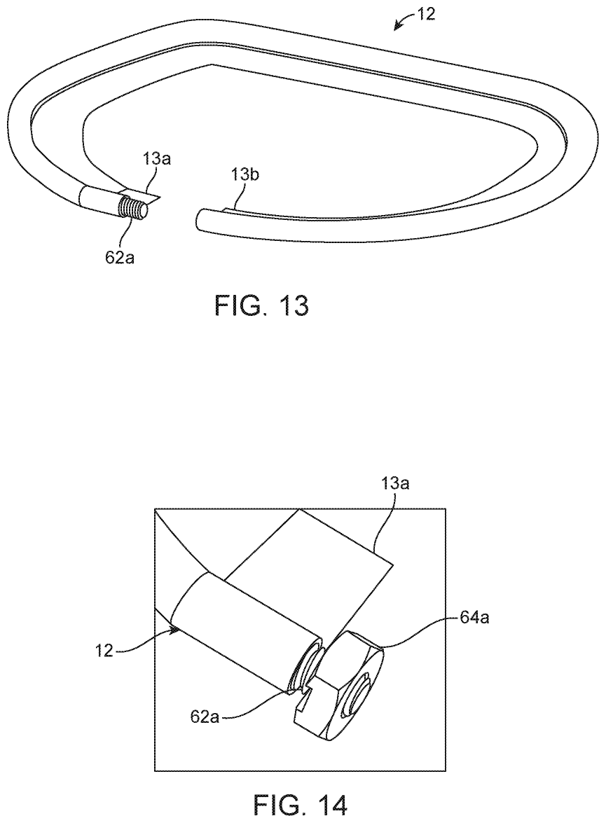

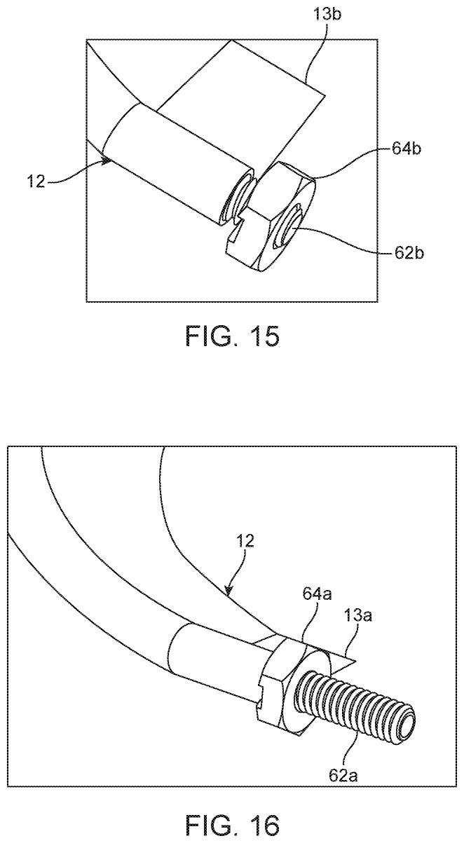

Yet another cinching device 60 is collectively illustrated in FIGS. 11-16. Each end 21a, 21b of the cinching member 20 includes a threaded shaft 62a, 62b on which a nut 64a, 64b is threadably engaged. In this embodiment, after the implant 12 is secured to the annulus 2, the cinching device 60 and other components of the cinching device 60 are supported within a catheter 61 and tracked over one or more guidewires (not shown) into a left atrium, through a septal hole to deliver the cinching device 60 adjacent one of the ends 13a, 13b of the implant 12, to one of the nuts 64a, 64b. In one example, the cinching member 20 has a central bore for a guidewire (or equivalent) to travel through. The distal portion of this guidewire is external to the patient, allowing the cinching catheter 60 to be tracked over this guidewire directly to nuts 64a, 64b. Once the cinching device 60 is connected to one nut 64a, 64b, an internal member 66 engaged with one nut 64a, 64b rotates to draw the threaded shaft 62a, 62b away from the respective implant end 13a, 13b until the desired amount of proximal tensioning, cinching or the like of the cinching member(s) 20 is achieved. The process can be repeated on the second threaded shaft 62a, 62b to cinch from the other side, as desired. Alternatively, both nuts 64a, 64b can be tightened at the same time. Once the implant 12 has been cinched sufficiently, the guidewire is removed.

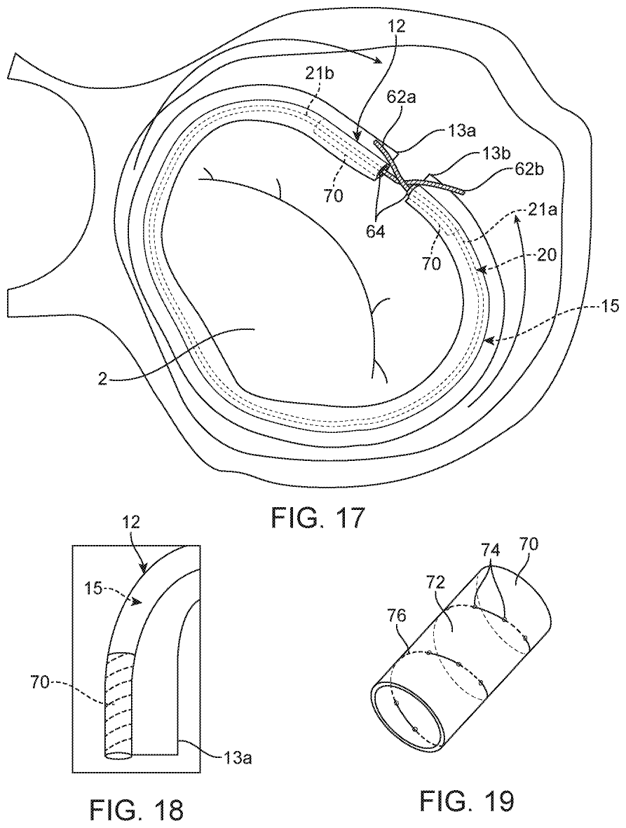

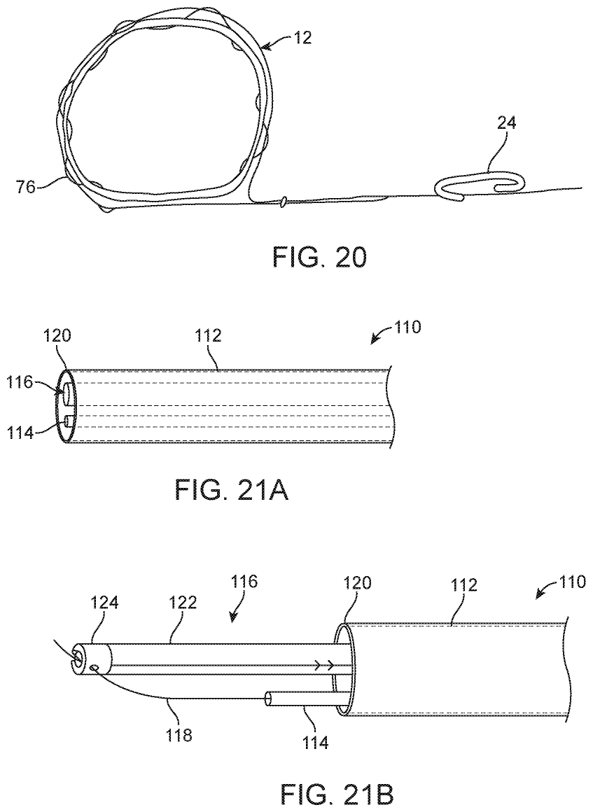

In some optional embodiments, as shown in FIGS. 17-19, the implant may include a support 70 to reinforce the implant during cinching. The support can be a tubular body 72 that reinforces first and second ends 13a, 13b of the implant 12, adjacent where the cinching member(s) exit the implant 12. The support 70 may have a greater stiffness with respect to the material of the implant 12. The support 70 can include one or more sewing apertures 74 provided in the body 72. In such embodiments, the lumen formed by the implant 12 may further be defined by a stich 76 extending through the body 72, and along a length of the implant 12 as shown in FIG. 20. The stich 76 can further assist to secure the support 70 to the implant 12.

Referring now in addition to FIGS. 21A-21F which illustrate an alternate delivery device 110 that can be used in systems and methods disclosed herein. In this example, the delivery device 110 includes a handle assembly (such as the handle assembly 26 of FIG. 1A, for example) supporting a guide catheter 112 having an implant catheter 114 and a steerable anchor catheter 116 slidably housed therein. The implant catheter 114 is separately articulable with respect to the anchor catheter 116. An implant 118 is slidably housed in the implant catheter 114. The implant 118 can include, for example, a straight wire, braided wire, cloth-covered wire or the like. As illustrated in FIG. 21A, the delivery device 110 includes a delivery arrangement in which the implant catheter 114 and anchor catheter 116 are housed entirely within the guide catheter 112. As shown in FIG. 21B, the delivery device 110 further includes a partially-deployed arrangement in which both the implant catheter 114 and the anchor catheter 116 extend distally out of a distal end 120 of the guide catheter 112. In this partially-deployed arrangement, the implant 118 is engaged with the anchor catheter 116. As best shown in FIG. 21B, the implant 118 may be threaded through the anchor catheter 116 as will be discussed in further detail below.

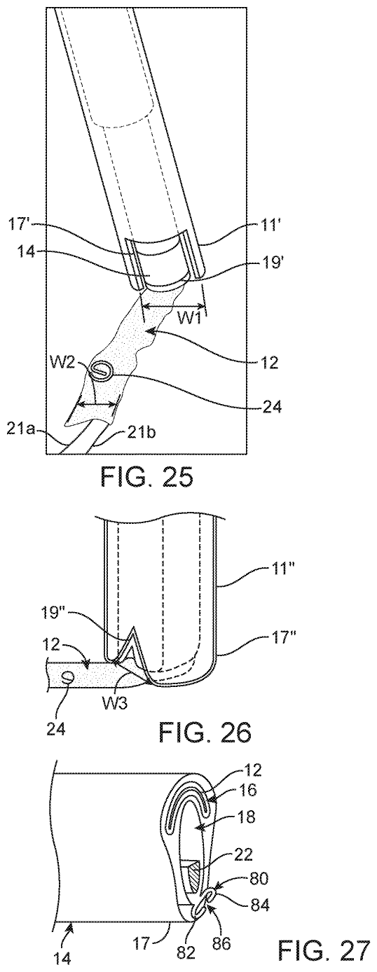

The anchor catheter 116 houses an anchor delivery apparatus, which is not visible in FIGS. 21A-21F but can be of any of the types disclosed herein. In non-limiting examples, the anchor delivery apparatus is model number SG-64, HG-16-62-28, SA-85, or HG-19-90-22 sold by Medtronic. Inc. of Minneapolis, Minnesota. In such an example, a rotational drive member drives a helical anchor through an internally threaded tip into the implant (e.g., implant 12 or 118) and the tissue. Generally, the anchor delivery apparatus is slidably housed within the anchor catheter 116. In one example, the anchor catheter 116 can include a drive shaft 122 terminating at collar 124. In one embodiment, the collar 124 includes an aperture 126 and a slot 128 that is positioned about 180 degrees (+/−5 degrees) from the aperture 126. The collar 124 serves to maintain alignment of the implant 118 with the anchor to be disposed by the anchor delivery apparatus within the anchor catheter 116 to ensure that the anchor 24 engages the implant 118 and accurately secures the implant 118 to the annulus 2 during use. For example, the implant 118 passes through the aperture 126, and then exits through the slot 128 so that the implant 118 extends across a passageway 130 formed by the collar 124. This allows the anchor delivery apparatus to track along a length of the implant 118 as anchors A are deployed around the annulus 2 while staying aligned with the implant 118. The collar 124 is best suited for an implant 118 being a straight wire, braided wire or the like. Other alternate collar configurations can be utilized as will be discussed in greater detail with respect to FIGS. 25-31. Once the first anchor 24 is deployed to secure the implant 118 to the annulus 2, the steering capabilities of the anchor delivery apparatus serve to align the implant 118 and the anchor delivery apparatus within the anchor catheter 116. In some examples, the anchor catheter 116 is removed after each anchor 24 is delivered to reload, but alternate examples could have the anchors A lined up for sequential deployment within the anchor delivery catheter 116.

The delivery device 110 is configured to be transitioned from the delivery arrangement (FIG. 21A) to the partially deployed arrangement (FIG. 21B) to a deployed arrangement (FIG. 21F). During this transition, at least a portion of the anchor catheter 116 is positioned outside of the guide catheter 112 and transitions from a linear arrangement (FIG. 21B) to a curved arrangement (FIG. 21F) so that the distal end 140 of the anchor catheter 116 is generally perpendicular (+/−5 degrees) from a longitudinal axis defined by both of the implant 118 and the implant catheter 114. In particular, the distal end 140 is curved such that the collar 124 is adjacent a distal end 142 of the implant catheter 114 so that the implant 118 can linearly extend from the implant catheter 114, through the collar 124. Actuation of the anchor catheter 116 can be achieved via selective tensioning of one or more pull wires 144, 146 embedded within or secured to a body 148 of the anchor catheter 116 and extending a long a length of the body 148 to the handle assembly. In one example, the pull wires 144, 146 are located approximately 180 degrees (+/−5 degrees) from each other. In this example, the first pull wire 144 extends to the collar 124 and the second pull wire 146 terminates proximal with respect to the first pull wire 144. The pull wires 144, 146 can optionally be actuated via the handle assembly. Control of the shape or arrangement of the anchor catheter 116 can also at least partially be controlled with material selection of the body 148. In one embodiment, the body 148 includes a distal section 150a and a proximal section 150b. The distal section 150a may be made of a more flexible material as compared to the proximal section 150b, which will increase the distal section's 150a propensity to bend upon proximal movement of the pull wire(s) 144, 146 as compared to the proximal section 150b. In such an example, the second pull wire 146 can terminate at a junction of the distal section 150a and the proximal section 150b. After each anchor 24 is deployed to anchor the implant 118 into the annulus 2, the anchor delivery apparatus is withdrawn into the anchor catheter 116 to take up another anchor. The anchor catheter remains inside the left atrium during this process. The steering (one plane) in the anchor catheter 116/anchor delivery apparatus coupled with the steering in the guide catheter 112 (one plane) provides access to all points along the annular path for application of the implant 118 (e.g., P1/A1 to P3/A3) to sequentially deliver and anchor the implant 118 around the annulus 2 in a similar configuration that which is shown in FIGS. 4-5M, for example. Once the implant 118 has been anchored to the mitral annulus 2 as desired, one or more cinching members can be pulled proximally or tensioned through the anchors A, along a central longitudinal axis of the delivery device 110, (in the case of the bare wire implant) or lumen(s) to resize the circumference of the implant 118 and annulus orifice. Such cinching can provide a reshaping of the implant and annulus attached thereto. Examples of some suitable cinching techniques are shown and discussed with respect to FIGS. 5A-16. It will be understood that the implant 118 is essentially a “cinching member” of the disclosure that is anchored to the annulus 2. The cinched length of the implant 118 can be secured using one of the locking mechanism generally disclosed with respect to the collets 46, 46′ of FIGS. 6A-7 or 9A-9B, for example. The anchors disclosed herein can have a variety of configurations.

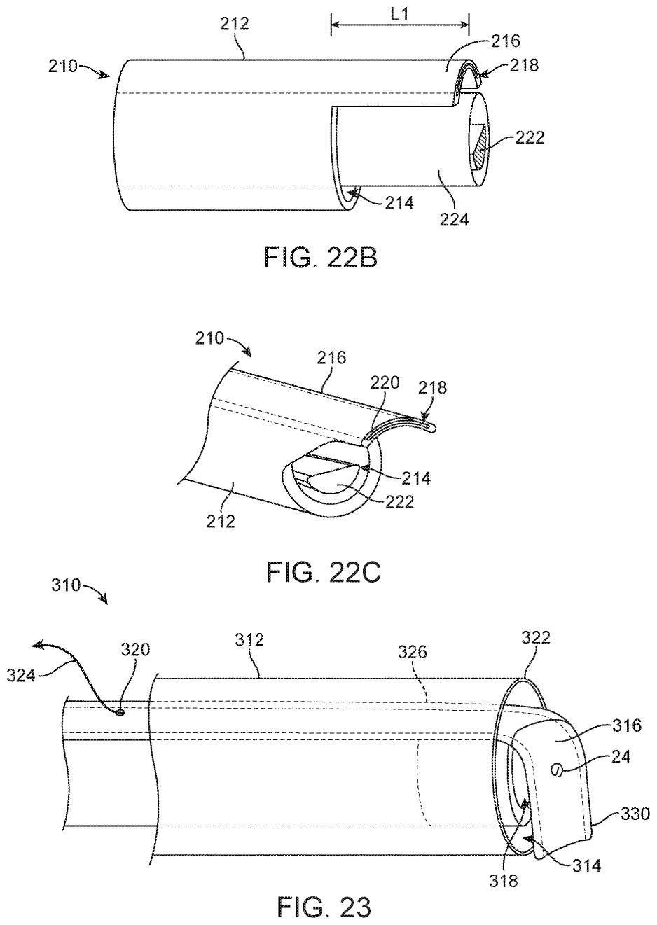

Referring also to FIGS. 22A-22C, which illustrate an alternate delivery device 210 that can be used with any of the delivery systems disclosed herein and used in an identical manner except as explicitly stated. In this embodiment, the delivery device 210 includes a catheter body 212 forming a main lumen 214. The body 212 further includes a protrusion 216 defining a first lumen 218 having an extruded crescent shape for receiving the implant 220. Various embodiments are made by forming the first lumen 218 and the main lumen 214 from a unitary piece of tubing so that the body 212 is integrally formed. In one example, the main lumen 214 and the first lumen 218 intersect at a proximal end of the protrusion 216. In one illustrative example, the protrusion 216 has a length L1 of ~1.5 cm. The implant 220 can be routed through the main lumen 214 to the first lumen 218 in a folded or flattened arrangement. In either the flattened or folded arrangement the implant 220 is in the arrangement on which it is to lay on the annulus. An anchor delivery apparatus 222 can be delivered through the main lumen 214 within an anchor catheter 224 (FIG. 22B) and then will exit the main lumen and the body 212 at a proximal end of the protrusion 216. In an alternate example, the anchor catheter 224 shown in FIG. 22B can be omitted as is shown in FIG. 22C. The anchor delivery apparatus 222 can be identical to any of those disclosed herein. With these embodiments, the implant 220 is dispensed from the first lumen 218 adjacent the anchor delivery apparatus 222. As with prior disclosed embodiments, the anchor delivery apparatus 222 can then sequentially dispense anchors to secure the implant around the annulus 2 as the implant 220 is dispensed from the delivery device 210 in a manner very similar to that of FIGS. 3A-4.

Referring in addition to FIG. 23, which illustrates an alternate delivery device 310 that can be used with any of the delivery systems disclosed herein and used in an identical manner except as explicitly stated. In this embodiment, the delivery device 310 includes a guide catheter 312 forming a main lumen 314 that houses an implant 316 and the anchor delivery apparatus 318. An optional outer catheter, such as catheter 11 of FIG. 1B, can also be provided as part of the delivery device 310 to sheath at least a portion of guide catheter 312. The implant 316 and anchor delivery apparatus 318 can be any of the types disclosed herein. In one example, the implant 316 (e.g., cloth implant) is draped over the anchor delivery apparatus 318 so that the implant 316 extends along a length of the anchor delivery apparatus 318 and contacts the outer surface of the anchor delivery apparatus 318. Optionally, the guide catheter 312 can be configured to be steerable in two planes and can also be torquable with an outer catheter (e.g., outer catheter 11 of FIG. 1B) that is steerable in one plane and torquable. The system can have a different combination of steering and torqueing capabilities between the outer catheter and guide catheter 312, as desired. Steering and torqueing capabilities can be accomplished with pull wire(s) and the handle assembly (e.g., handle assembly 26 of FIG. 1A), in the manner disclosed above, for example. In this embodiment, an aperture 320 can be provided in the guide catheter 312, approximately 6 mm from a distalmost edge 322 of the guide catheter 312, where a tensioning member 324, such as a suture or the like, is attached (via backstitch or the like) to the implant 316. The tensioning member 324 can be used to tension the implant 316 when the anchor delivery apparatus 318 is inserted into the guide catheter 312. When the tensioning member 324 is pulled proximally, the tensioning member 324 forces the implant 316 against the outer surface 326 of the guide catheter 312 in a tensioned fashion, reducing implant bunching and allowing the anchor delivery apparatus 318 to pass through the guide catheter 312 unobstructed. Tensioning member 324 adjustment can be controlled with the handle assembly, for example. The implant 316 is loaded into the guide catheter 312 with a distal end 330 of the implant 316 covering the distal tip of the anchor delivery apparatus 318. To temporarily secure the implant 316 to the anchor delivery apparatus 318, a first anchor 24 maintained by the anchor delivery apparatus 318 that is partially-deployed though the implant 316 as is shown. Once the first anchor 24 is in position and secured to the annulus, the tensioning member 324 is pulled proximally to tension the implant 316 and the first anchor 24 acts to counter tension in the tensioning member 324. The anchor delivery apparatus 318 can be utilized to delivery several anchors into the implant 316 along the annulus in a similar manner to that shown in FIGS. 3A-4 or in any other manner of the disclosure. Once the implant 316 has been fully anchored to the annulus, any cinching member(s) provided within the implant as disclosed herein (e.g., see can be pulled proximally or tensioned through the implant to decrease the interior dimensions of the implant and the annulus secured thereto. This cinched length can be secured using a locking mechanism as disclosed herein, for example.

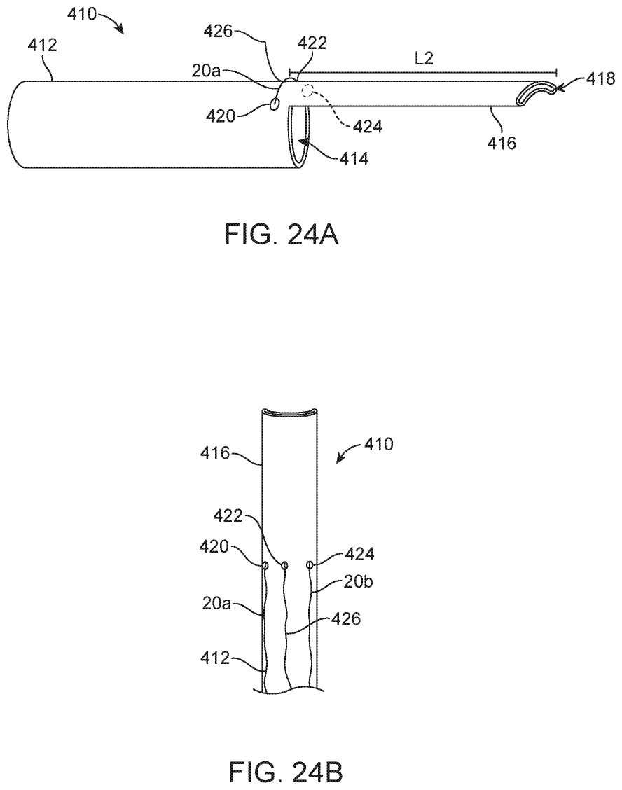

Referring in addition to FIGS. 24-24B, which illustrates an alternate guide catheter 410 that can be used with the delivery systems and methods disclosed herein. In this embodiment, the guide catheter 410 includes a tubular body 412 defining a main lumen 414. Extending from the body 412 is a protrusion 416 having a crescent shaped lumen 418. In one example, the protrusion 416 and the lumen 418 extend a length that is at least a length L2 of the implant (not shown, see also FIG. 4, for example). In one embodiment, the length L2 is about 4 inches to about 8 inches and the implant is a cloth implant. In one example, the length L2 is ~5 inches. The main lumen 414 includes a generally circular cross-section. Proximate an intersection of the protrusion 416 and the main lumen 414, the body 412 includes three apertures 420, 422, 424. One aperture 422 receives an elongated member 426 (e.g., suture or the like) of the implant for selective tensioning the implant as discussed with respect to FIG. 23 and two apertures 420, 424 for receiving one or more respective cinching members of the implant. The apertures 420, 424 are therefore aligned with respective lumens in the implant through which the cinching member(s) are housed (see also, FIG. 7 and related disclosure). The elongated member 426 and cinching member(s) are directed together to though the apertures 420, 424 in the guide catheter 410 to the handle assembly (e.g., handle assembly 26 of FIG. 1A), for example.

Optionally, any of the catheters disclosed herein can be modified to include a guide configured to align the implant with the anchor delivery apparatus as the implant exits the delivery device. In the example of FIG. 25, a catheter 11′ (substantially similar to catheter 11 of FIG. 1B) includes a distal end 17 defining a rectangular cutout or window 19. The cutout 19 is configured to have a width W1 that is slightly larger (i.e. about 3 to about 15% larger) than a width W2 of the implant 12. Similarly, in the example of FIG. 26, a distal end 17″ of the catheter 11″ can include a triangular shaped cutout or window 19″ that receives and aligns the implant 12. Similarly, a maximum width W3 of the window 19″ is slightly larger (i.e. about 3 to about 15% larger) than the width W2 of the implant 12. In the example of FIG. 27, the distal end 17 of the catheter 11 of FIG. 1B can have a guide 80 attached thereto. The guide 80 can be C-shaped having opposing arms 82 define a gap or opening 84 in which the implant 12 can be received and aligned. As the device is clocked around the annulus 2, the combination of the guide 80 and the implant lumen prevent the implant from covering a distal face of the anchor delivery apparatus, allowing for more accurate anchor deployment. It is to be understood that the guides of FIGS. 25-27 can be applied to any of the catheters disclosed herein for use in aligning the implant as it exits the device.

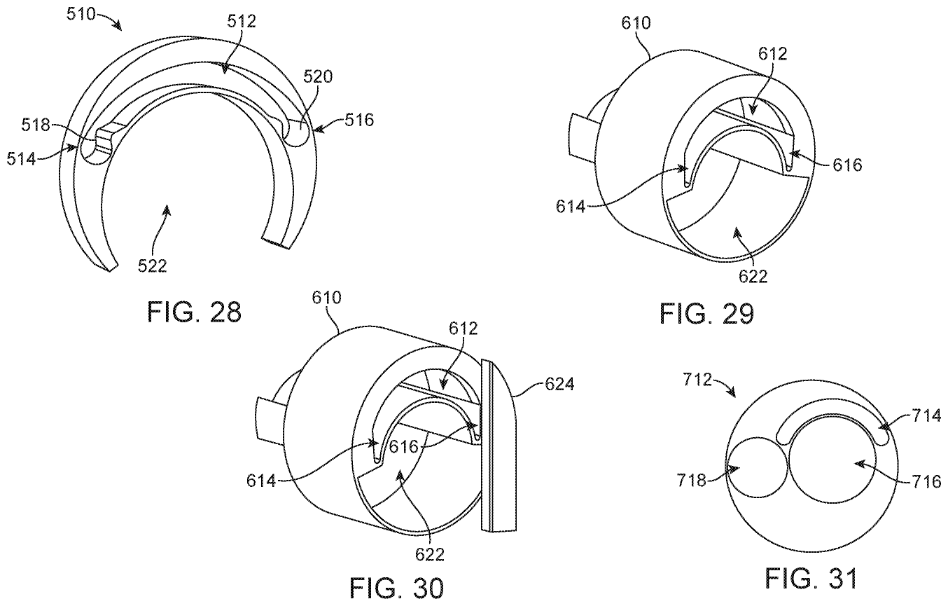

Referring now in addition to FIG. 28, FIG. 28 illustrates a cross-section of an alternate lumen 512 that can be incorporated into a catheter or catheter protrusion of the disclosure to house and maintain position of the implant (e.g., implant 12). In this example, the lumen 512 includes first and second ends 514, 516 including grooves 518, 520. Each groove 518, 520 can receive one or more cinching members (e.g., cinching member 20) within the folded implant (see also FIGS. 4 and 7). During cinching, the cinching members can travel within one respective groove 518, 520 to maintain alignment of the cinching members and the implant as a whole as the implant travels through lumen 512 to be dispensed. Alternatively, the lumen 512 can be part of a collar 510 that can be secured to a distal end of any of the guide catheters of the disclosure for disposing a fabric implant having one or more folds or cinching members. In such an example, the collar 510 can include a main lumen 522 through which the anchor catheter/anchor delivery apparatus can pass. Collar 510 is particularly useful with the embodiment of FIG. 23.

FIG. 29 illustrates an alternate lumen 612 configuration for housing and maintaining position of the implant while the implant is within the device. Alternatively the lumen 612 of FIG. 30 can be provided within a collar 610 for securing to the distal end of any of the guide catheters of the disclosure for disposing a fabric implant (e.g., implant 12). In this example, the lumen 612 includes first and second ends 614, 616, each of which are tapered. The collar 610 can further define a main lumen 622 through which the anchor catheter/anchor delivery apparatus can pass.

FIG. 30 illustrates the collar 610 of FIG. 29 including an optional bumper 624 provided at the second end 6161 of the lumen 612. The bumper 624 is positioned between the implant and the annulus, forcing the implant to stay on the annulus as the delivery catheter is steered around the annulus. Without this bumper 264, the implant can take the “short route” between two adjacent anchoring points. If the bumper 624 or the like is provided, the implant must take the “long path” (i.e. the arc length of the annulus as compared to a shortest, straight distance between two points), which keeps delivery of the implant on the annulus. The bumper 624 further maintains alignment of the implant with the anchor delivery apparatus after the implant has exited a lumen 612 of the collar 610.

Referring in addition to FIG. 31, which illustrates an embodiment of a guide catheter 712 having a first lumen 714 to house the implant, a second lumen 716 to house the anchor catheter and/or the anchor delivery apparatus and a third lumen 718 to house an intra-cardiac echocardiography catheter or other imaging apparatus, for example. It will be understood that any of the guide catheters disclosed herein can be similarly configured to accommodate the lumen 718, as desired.

Various non-limiting methods of the disclosure can be described as follows. Methods of repairing a valve annulus can include providing a system including a guide catheter. An implant is positioned at least partially within the guide catheter and an anchor delivery apparatus is positioned within the guide catheter. The method includes directing a distal end of the guide catheter to a valve annulus, advancing the implant at least partially out of the guide catheter and securing the implant to a valve annulus with the anchor delivery apparatus. The method further includes advancing the implant out of the guide catheter and further securing the implant to the valve annulus with the delivery apparatus. The system can include any features of the present disclosure. In some methods, the implant is tensioned as the implant is advanced out of the guide catheter. Optionally, the implant includes an elongated cinching member, the method further comprising the step of tensioning the elongated cinching member to reshape the valve annulus after the implant is fully advanced out of the guide catheter. In some examples, the elongated cinching member includes a first end and a second end, wherein the first and second ends are both tensioned during the step of tensioning. In one example, the guide catheter includes a first lumen having first and second grooves, wherein the elongated cinching member is routed through the first and second grooves. In one method, the method further includes symmetrically reshaping the implant with the elongated cinching member. In various methods, the elongated cinching member is tensioned with a cinching catheter having a collet that is engaged with the elongated cinching member. In some examples, the elongated cinching member is tensioned with a cinching catheter having a nut that is engaged with the elongated cinching member. In one example, the guide catheter houses an anchor delivery catheter in which the anchor delivery apparatus is positioned, the method further comprising the step of advancing the anchor delivery catheter out of the guide catheter and bending the anchor delivery catheter to position the anchor delivery apparatus. In various methods, the step of securing includes deploying a plurality of anchors with the anchor delivery apparatus. In such methods, the implant is optionally selected from the group consisting of a wire implant and a fabric implant.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method of repairing a valve annulus, the method comprising:
providing a system including a guide catheter, an implant positioned at least partially within the guide catheter and an anchor delivery apparatus within the guide catheter;
directing a distal end of the guide catheter to a valve annulus;
advancing the implant at least partially out of the guide catheter;
securing the implant to a valve annulus with the anchor delivery apparatus;
further advancing the implant out of the guide catheter; and
further securing the implant to the valve annulus with the delivery apparatus.

2. The method of claim 1, wherein the implant is tensioned as the implant is advanced out of the guide catheter.

3. The method of claim 1, wherein the implant includes an elongated cinching member, the method further comprising the step of tensioning the elongated cinching member to reshape the valve annulus after the implant is fully advanced out of the guide catheter.

4. The method of claim 3, wherein the elongated cinching member includes a first end and a second end, wherein the first and second ends are both tensioned during the step of tensioning.

5. The method of claim 3, wherein the guide catheter includes a first lumen having first and second grooves, wherein the elongated cinching member is routed through the first and second grooves.

6. The method of claim 3, further comprising symmetrically reshaping the implant with the elongated cinching member.

7. The method of claim 3, wherein the elongated cinching member is tensioned with a cinching catheter having a collet that is engaged with the elongated cinching member.

8. The method of claim 3, wherein the elongated cinching member is tensioned with a cinching catheter having a nut that is engaged with the elongated cinching member.

9. The method of claim 1, wherein the guide catheter houses an anchor delivery catheter in which the anchor delivery apparatus is positioned, the method further comprising the step of advancing the anchor delivery catheter out of the guide catheter and bending the anchor delivery catheter to position the anchor delivery apparatus.

10. The method of claim 1, wherein the step of securing includes deploying a plurality of anchors with the anchor delivery apparatus.

11. The method of claim 1, wherein the implant is selected from the group consisting of a wire implant and a fabric implant.
